SEQUENCE LISTING (26 sequences)

SEQ ID NO: 1 - protein, 1073 aa, Artificial Sequence
  SFTS GnGc protein
MMKVIWFSSLICLVIQCSGDSGPIICAGPIHSNKSASIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKACKTSGSCRGDMQFCKVAGCEHGEEASDAKCRCSLVHKPGEVVVSYGGMRVRPKCYGFSRMMATLEVNPPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKKWLFILIVILLGYAGLMLITNVLKAIGVWGSWVIAPVKLMFAIIKKLMRTVSCLMGKLMDRGRQVIHEEIGENGEGNQDDVRIEMARPRRVRHWMYSPVILTILAIGLAEGCDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVPKFVDGSYMQTYHSTVPTGANIPSPTDWLNALFGNGLSRWILGVIGVLLGGLALFFLIMSLFKLGTKQVFRSRTKLA

SEQ ID NO: 2 - protein, 245 aa, Artificial Sequence
  SFTS nucleoprotein (NP)
MSEWSRIAVEFGEQQLNLSELEDFARELAYEGLDPALIIKKLKETGGDDWVKDTKFIIVFALTRGNKIVKASGKMSNSGSKRLMALQEKYGLVERAETRLSITPVRVAQSLPTWTCAAAAALKEYLPVGPAVMNLKVENYPPEMMCMAFGSLIPTAGVSEATTKTLMEAYSLWQDAFTKTINVKMRGASKTEVYNSFRDPLHAAVNSVFFPNDVRVKWLKAKGILGPDGVPSRAAEVAAAAYRNL

SEQ ID NO: 3 - protein, 293 aa, Artificial Sequence
  SFTS nonstructural (NS) protein
MSLSKSSNVDLKSVAMNANTVRLEPSLGEYPTLRRDLVECSCSVLTLSMVKRMGKMTNTVWLFGNPKNPLHQLEPGLEQLLDMYYKDMRCYSQRELSALRWPSGKPSVWFLQAAHMFFSIKNSWAMETGKENWRGLFHRITKGQKYLFEGDMILDSLEAIEKRRLRLGLPEILITGLSPILDVALLQIESLARLRGMSLNHHLFTSSSLRKPLLDCWDFFIPVRKKKTDGSYSVLDEDDEPGALQGYPHLMAHYLNRCPFHNLIRFDEELRTAALNTIWGRDWPAIGDLPKEV

SEQ ID NO: 4 - DNA, 3222 bp, Artificial Sequence
  polynucleotide encoding SFTS GnGc protein
atgatgaaagtgatttggttctcctctctgatttgtctggtcattcagtgtagcggggattctggacctattatctgtgctgggccaatccacagcaacaagagcgcctccatcccccacctgctgggctactccgagaagatctgccagatcgaccgcctgatccacgtgagctcctggctgcggaaccacagccagttccagggatacgtgggacagaggggaggccgcagccaggtgtcctactatccagccgagaattcttatagcagatggtccggcctgctgtctccatgtgacgcagattggctgggcatgctggtggtgaagaaggccaagggctctgatatgatcgtgcctggcccaagctacaagggcaaggtgttctttgagcggcccaccttcgacggatatgtgggatggggatgcggatctggcaagagcaggacagagtccggcgagctgtgcagcagcgattctggcacctcctctggcctgctgcctagcgatcgcgtgctgtggatcggcgacgtggcatgccagccaatgacacccatccctgaggagacattcctggagctgaagtccttctctcagagcgagtttcctgatatctgcaagatcgacggcatcgtgttcaatcagtgtgagggcgagagcctgccacagccctttgatgtggcctggatggacgtgggccactcccacaagatcatcatgcgggagcacaagaccaagtgggtgcaggagagctcctctaaggacttcgtgtgctacaaggagggcacaggcccatgttccgagtctgaggagaaggcctgcaagaccagcggctcctgtagaggcgatatgcagttttgcaaggtggcaggatgtgagcacggagaggaggcctctgacgccaagtgcaggtgtagcctggtgcacaagccaggagaggtggtggtgtcttacggaggaatgcgggtgcggcccaagtgctatggcttcagcagaatgatggccacactggaggtgaacccccctgagcagaggatcggccagtgcaccggctgtcacctggagtgtatcaatggcggcgtgaggctgatcaccctgacaagcgagctgaagtccgccacagtgtgcgccagccacttctgtagctccgccacatctggcaagaagagcaccgagatccagtttcactctggcagcctggtgggcaagaccgcaatccacgtgaagggcgccctggtggatggcacagagttcacctttgagggctcctgcatgttcccagacggctgtgatgccgtggactgcaccttctgtagagagtttctgaagaacccacagtgctaccccgccaagaagtggctgtttatcctgatcgtgatcctgctgggctatgccggcctgatgctgatcacaaatgtgctgaaggccatcggcgtgtggggatcttgggtcatcgcacccgtgaagctgatgtttgccatcatcaagaagctgatgaggaccgtgtcctgcctgatgggcaagctgatggacaggggccggcaggtcatccacgaggagatcggcgagaacggcgagggcaatcaggacgatgtgcggatcgagatggccagacctcggagagtgaggcactggatgtacagcccagtgatcctgacaatcctggcaatcggcctggcagagggatgcgatgagatggtgcacgccgactccaagctggtgtcttgccgccagggctccggcaacatgaaggagtgcgtgaccacaggccgggccctgctgcctgcagtgaatccaggccaggaggcatgtctgcacttcaccgcaccaggctcccctgactctaagtgcctgaagatcaaggtgaagcgcatcaacctgaagtgtaagaagtctagctcctattttgtgcccgatgcccggagcagatgcacatccgtgaggcgctgtagatgggcaggcgactgccagagcggctgtccaccccacttcacctccaattcttttagcgacgattgggccggcaagatggatagagcaggcctgggcttcagcggatgctccgacggatgtggaggagcagcatgcggatgtttcaacgcagccccctcctgcatcttttggaggaagtgggtggagaatcctcacggcatcatctggaaggtgagcccatgtgcagcatgggtgccctccgccgtgatcgagctgacaatgccatccggagaggtgcgcaccttccaccccatgtctggcatccctacacaggtgtttaagggcgtgagcgtgacctacctgggcagcgatatggaggtgtccggcctgaccgacctgtgcgagatcgaggagctgaagtccaagaagctggccctggcaccttgtaaccaggcaggaatgggagtggtgggcaaggtcggcgagatccagtgctctagcgaggagagcgcccgcacaatcaagaaggatggctgtatctggaacgcagacctggtgggaatcgagctgagggtggacgatgccgtgtgctactctaagatcaccagcgtggaggccgtggccaattattccgccatccctaccacaatcggcggcctgcggttcgagagatctcacgatagccagggcaagatctccggctctccactggacatcaccgccatccgcggcagcttttccgtgaactacaggggcctgcgcctgtctctgagcgagatcaccgccacatgcaccggcgaggtgacaaacgtgagcggctgctattcttgtatgaccggcgccaaggtgtctatcaagctgcactcctctaagaacagcacagcccacgtgcggtgtaagggcgatgagacagccttctccgtgctggagggcgtgcactcttatacagtgtccctgtcttttgatcacgccgtggtggacgagcagtgccagctgaactgtggcggccacgagagccaggtgaccctgaagggcaatctgatcttcctggatgtgccaaagtttgtggacggcagctacatgcagacatatcactccacagtgcccaccggcgccaatatcccttccccaaccgactggctgaacgccctgttcggcaatggcctgtctaggtggatcctgggcgtgatcggcgtgctgctgggaggcctggccctgttctttctgatcatgtctctgttcaaactgggcactaagcaggtctttcggtcccgcacaaaactggcttaa

SEQ ID NO: 5 - DNA, 735 bp, Artificial Sequence
  polynucleotide encoding SFTS nucleoprotein (NP)
atgtcggagtggtccaggattgcagtggagtttggtgagcagcagctcaatttgtctgagcttgaggatttcgcgagagaactggcctatgaaggccttgaccctgctttgatcatcaagaagctgaaggagacaggtggagatgattgggtgaaggatacaaagttcatcattgtctttgccctgactcgaggcaacaagatcgtcaaggcatcagggaaaatgtcaaactcagggtccaagaggttgatggcactccaagagaaatatggactggttgagagggcagagaccaggctctcaatcactcctgtgagggttgcgcagagccttcccacttggacatgtgctgcagcagcagccttgaaggagtatctccctgtggggccagccgtcatgaacctgaaggtcgagaattatccccctgagatgatgtgcatggcctttggatccctgattccaactgcgggggtatctgaagccacaaccaagaccctgatggaggcctactctctgtggcaagatgccttcaccaagactatcaatgtgaagatgcgcggagccagcaagacagaagtttacaactccttcagggaccctcttcatgctgctgtgaactctgtcttctttcccaatgatgttcgggtaaagtggctgaaggccaaggggatccttggcccagatggggtccccagcagagctgctgaggttgctgctgctgcttacagaaacctg

SEQ ID NO: 6 - DNA, 882 bp, Artificial Sequence
  polynucleotide encoding SFTS nonstructural (NS) protein
atgagcctgagtaagagcagcaatgtggatctgaaaagcgtcgcaatgaatgccaacaccgtccgcctggaaccatcactgggggaataccctaccctgaggcgggacctggtggagtgcagctgttccgtgctgacactgtctatggtgaagaggatgggcaagatgaccaacacagtgtggctgttcggcaaccccaagaatcctctgcaccagctggagccaggcctggagcagctgctggacatgtactataaggatatgaggtgctactctcagagggagctgagcgccctgaggtggccctccggcaagcccagcgtgtggtttctccaggccgcccacatgttctttagcatcaagaactcctgggctatggagacaggcaaggagaattggcggggcctgttccacagaatcacaaagggccagaagtatctgtttgagggcgacatgatcctggatagcctggaggcaatcgagaagaggcgcctgaggctgggcctgccagagatcctgatcaccggcctgtcccctatcctggacgtggccctgctccagatcgagtccctggcccggctgagaggcatgtctctgaatcaccacctgttcaccagctcctctctgcggaagccactgctggactgctgggatttctttatccccgtgcggaagaagaagaccgacggctcttatagcgtgctggatgaggacgatgagcccggcgccctccagggctacccacacctgatggcccactatctgaacaggtgtcccttccacaatctgatcaggtttgatgaggagctgcgcacagccgccctgaataccatctgggggcgggactggcctgctatcggcgacctgcctaaggaagtgtaa

SEQ ID NO: 7 - protein, 25 aa, Artificial Sequence
  tPA signal sequence
MDAMKRGLCCVLLLCGAVFVSPSHA

SEQ ID NO: 8 - DNA, 75 bp, Artificial Sequence
  polynucleotide encoding tPA signal sequence
atggacgccatgaagagaggcctgtgctgcgtgctgctgctgtgcggcgccgtgttcgtgagccccagccacgcc

SEQ ID NO: 9 - protein, 156 aa, Artificial Sequence
  Flt3L
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAP

SEQ ID NO: 10 - DNA, 468 bp, Artificial Sequence
  polynucleotide encoding Flt3L
acccaggactgcagcttccagcacagccccatcagcagcgacttcgccgtgaagatcagagagctgagcgactacctgctgcaggactaccccgtgaccgtggccagcaacctgcaggacgaggagctgtgcggcggcctgtggagactggtgctggcccagagatggatggagagactgaagaccgtggccggcagcaagatgcagggcctgctggagagagtgaacaccgagatccacttcgtgaccaagtgcgccttccagcctccccccagctgcctgaggttcgtgcagaccaacatcagcagactgctgcaggagaccagcgagcagctggtggccctgaagccctggatcaccagacagaacttcagcagatgcctggagctgcagtgccagcccgacagcagcaccctgccccctccctggagccccagacccctggaggccaccgctcccacagcccct

SEQ ID NO: 11 - protein, 505 aa, Artificial Sequence
  Gn peptide
SGPIICAGPIHSNKSASIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKACKTSGSCRGDMQFCKVAGCEHGEEASDAKCRCSLVHKPGEVVVSYGGMRVRPKCYGFSRMMATLEVNPPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKKTNVLKAKKLMRTVSCLMGKLMDRGRQVIHEEIGENGEGNQDDVRIEMARPRRVRHWMYSPVILTILAIGLAEG

SEQ ID NO: 12 - DNA, 1518 bp, Artificial Sequence
  polynucleotide encoding Gn peptide
tccggccctatcatctgcgccggcccaatccactctaacaagtctgccagcatccctcacctgctgggctactccgagaagatctgtcagatcgatcgcctgatccacgtgagctcctggctgcggaaccactctcagttccagggatacgtgggacagaggggaggccgcagccaggtgtcctactatccagccgagaattcctattctcgctggtccggactgctgtctccatgcgacgcagattggctgggcatgctggtggtgaagaaggccaagggcagcgatatgatcgtgccaggcccctcctacaagggcaaggtgttctttgagcgcccaaccttcgacggatatgtgggatggggatgcggatctggcaagagccggaccgagtctggcgagctgtgcagcagcgatagcggcacatcctctggcctgctgcctagcgatagagtgctgtggatcggcgacgtggcatgccagccaatgacccctatcccagaggagacatttctggagctgaagagcttctcccagtctgagtttccagacatctgcaagatcgacggcatcgtgttcaatcagtgtgagggcgagtctctgccccagccttttgatgtggcctggatggacgtgggccacagccacaagatcatcatgagggagcacaagaccaagtgggtgcaggagagctcctctaaggacttcgtgtgctataaggagggaaccggaccatgtagcgagtccgaggagaaggcctgcaagacatctggcagctgtagaggcgatatgcagttttgcaaggtggcaggatgtgagcacggagaggaggcctccgacgcaaagtgcaggtgttctctggtgcacaagccaggagaggtggtggtgagctacggcggaatgagggtgcgccctaagtgctatggcttctccagaatgatggccaccctggaggtgaacccccctgagcagaggatcggccagtgcacaggctgtcacctggagtgtatcaatggcggcgtgcggctgatcaccctgacaagcgagctgaagtccgccaccgtgtgcgccagccacttctgtagctccgccacctctggcaagaagagcacagagatccagtttcactccggctctctggtgggcaagacagccatccacgtgaagggcgccctggtggatggcaccgagttcacatttgagggcagctgcatgtttcccgacggctgtgatgccgtggactgcaccttctgtcgcgagtttctgaagaacccacagtgctaccccgccaagaagaccaatgtgctgaaggccaagaagctgatgcggacagtgtcctgtctgatgggcaagctgatggaccggggccggcaggtcatccacgaggagatcggcgagaacggcgagggcaatcaggacgatgtgcggatcgagatggccagaccccggagagtgaggcactggatgtattcccctgtgatcctgacaatcctggccatcggcctggccgagggctaa

SEQ ID NO: 13 - DNA, 576 bp, Artificial Sequence
  CMV promoter
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 14 - protein, 488 aa, Artificial Sequence
  Gc peptide
CDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVPKFVDGSYMQTYHSTVPTGANIPSPTDWLNALFGNGLSRKLGTKQVFRSRTKLA

SEQ ID NO: 15 - DNA, 1467 bp, Artificial Sequence
  polynucleotide encoding Gc peptide
tgcgacgagatggtgcacgccgatagcaagctggtgtcctgcaggcagggcagcggcaacatgaaggagtgcgtgaccacaggccgcgccctgctgcccgccgtgaatcctggacaggaggcatgtctgcacttcaccgcacctggaagcccagactccaagtgcctgaagatcaaggtgaagaggatcaacctgaagtgtaagaagagctcctcttactttgtgcccgacgccaggtctcgctgcacaagcgtgcggagatgtagatgggcaggcgattgccagagcggatgtccacctcacttcacctccaattcttttagcgacgattgggcaggcaagatggaccgggcagggctggggttcagcggatgcagcgatggatgtggaggagcagcatgcggatgtttcaacgcagccccctcctgcatcttttggagaaagtgggtggagaatcctcacggcatcatctggaaggtgtctccttgtgcagcatgggtgccaagcgccgtgatcgagctgacaatgccaagcggagaggtgcggaccttccacccaatgtccggcatccccacacaggtgtttaagggcgtgtccgtgacctatctgggctctgacatggaggtgagcggcctgacagatctgtgcgagatcgaggagctgaagtctaagaagctggccctggccccttgtaaccaggcaggaatgggagtggtgggcaaggtcggcgagatccagtgcagctccgaggagagcgccaggacaatcaagaaggacggctgtatctggaacgccgatctggtgggcatcgagctgagagtggacgatgccgtgtgctactccaagatcacctctgtggaggccgtggccaattattccgccatccctaccacaatcggaggactgaggttcgagcggagccacgactctcagggcaagatctccggctctcccctggacatcaccgccatcaggggcagcttttccgtgaactaccggggcctgagactgtctctgagcgagatcaccgccacatgcaccggcgaggtgacaaatgtgagcggctgctattcctgtatgaccggcgccaaggtgtccatcaagctgcactctagcaagaactctacagcccacgtgcgctgtaagggcgacgagaccgccttctccgtgctggagggcgtgcactcttacacagtgtccctgtcttttgaccacgccgtggtggatgagcagtgccagctgaactgtggcggccacgagagccaggtgaccctgaagggcaatctgatcttcctggacgtgccaaagtttgtggatggcagctacatgcagacatatcactccacagtgcccaccggcgccaatatcccatcccccaccgattggctgaacgccctgttcggcaatggcctgtctcggaagctgggcacaaagcaggtgtttcggagcagaaccaagctggcctaa

SEQ ID NO: 16 - DNA, 566 bp, Artificial Sequence
  IRES
ccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgata

SEQ ID NO: 17 - protein, 551 aa, Artificial Sequence
  NS-NP fusion protein
MSEWSRIAVEFGEQQLNLSELEDFARELAYEGLDPALIIKKLKETGGDDWVKDTKFIIVFALTRGNKIVKASGKMSNSGSKRLMALQEKYGLVERAETRLSITPVRVAQSLPTWTCAAAAALKEYLPVGPAVMNLKVENYPPEMMCMAFGSLIPTAGVSEATTKTLMEAYSLWQDAFTKTINVKMRGASKTEVYNSFRDPLHAAVNSVFFPNDVRVKWLKAKGILGPDGVPSRAAEVAAAAYRNLGSGSGSGSGSGRAMSLSKSSNVDLKSVAMNANTVRLEPSLGEYPTLRRDLVECSCSVLTLSMVKRMGKMTNTVWLFGNPKNPLHQLEPGLEQLLDMYYKDMRCYSQRELSALRWPSGKPSVWFLQAAHMFFSIKNSWAMETGKENWRGLFHRITKGQKYLFEGDMILDSLEAIEKRRLRLGLPEILITGLSPILDVALLQIESLARLRGMSLNHHLFTSSSLRKPLLDCWDFFIPVRKKKTDGSYSVLDEDDEPGALQGYPHLMAHYLNRCPFHNLIRFDEELRTAALNTIWGRDWPAIGDLPKEV

SEQ ID NO: 18 - DNA, 1656 bp, Artificial Sequence
  polynucleotide encoding NS-NP fusion protein
atgtcggagtggtccaggattgcagtggagtttggtgagcagcagctcaatttgtctgagcttgaggatttcgcgagagaactggcctatgaaggccttgaccctgctttgatcatcaagaagctgaaggagacaggtggagatgattgggtgaaggatacaaagttcatcattgtctttgccctgactcgaggcaacaagatcgtcaaggcatcagggaaaatgtcaaactcagggtccaagaggttgatggcactccaagagaaatatggactggttgagagggcagagaccaggctctcaatcactcctgtgagggttgcgcagagccttcccacttggacatgtgctgcagcagcagccttgaaggagtatctccctgtggggccagccgtcatgaacctgaaggtcgagaattatccccctgagatgatgtgcatggcctttggatccctgattccaactgcgggggtatctgaagccacaaccaagaccctgatggaggcctactctctgtggcaagatgccttcaccaagactatcaatgtgaagatgcgcggagccagcaagacagaagtttacaactccttcagggaccctcttcatgctgctgtgaactctgtcttctttcccaatgatgttcgggtaaagtggctgaaggccaaggggatccttggcccagatggggtccccagcagagctgctgaggttgctgctgctgcttacagaaacctgggatcaggcagtggttcaggatcaggtagtgggcgcgccatgagcctgagtaagagcagcaatgtggatctgaaaagcgtcgcaatgaatgccaacaccgtccgcctggaaccatcactgggggaataccctaccctgaggcgggacctggtggagtgcagctgttccgtgctgacactgtctatggtgaagaggatgggcaagatgaccaacacagtgtggctgttcggcaaccccaagaatcctctgcaccagctggagccaggcctggagcagctgctggacatgtactataaggatatgaggtgctactctcagagggagctgagcgccctgaggtggccctccggcaagcccagcgtgtggtttctccaggccgcccacatgttctttagcatcaagaactcctgggctatggagacaggcaaggagaattggcggggcctgttccacagaatcacaaagggccagaagtatctgtttgagggcgacatgatcctggatagcctggaggcaatcgagaagaggcgcctgaggctgggcctgccagagatcctgatcaccggcctgtcccctatcctggacgtggccctgctccagatcgagtccctggcccggctgagaggcatgtctctgaatcaccacctgttcaccagctcctctctgcggaagccactgctggactgctgggatttctttatccccgtgcggaagaagaagaccgacggctcttatagcgtgctggatgaggacgatgagcccggcgccctccagggctacccacacctgatggcccactatctgaacaggtgtcccttccacaatctgatcaggtttgatgaggagctgcgcacagccgccctgaataccatctgggggcgggactggcctgctatcggcgacctgcctaaggaagtgtaa

SEQ ID NO: 19 - DNA, 527 bp, Artificial Sequence
  RSV promoter
cttggaggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcag

SEQ ID NO: 20 - protein, 215 aa, Artificial Sequence
  mIL-12A peptide
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 21 - protein, 336 aa, Artificial Sequence
  mIL-12B peptide
MGHQQLTISWFAIVLLVSPLMASMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYEQYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 22 - DNA, 768 bp, Artificial Sequence
  polynucleotide encoding mIL-12A peptide
atgtgtctcccaaggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgaaaggctcaaggccctctgccacagcgccctcctcacacagatagga

SEQ ID NO: 23 - DNA, 1011 bp, Artificial Sequence
  polynucleotide encoding mIL-12B peptide
atgggtcaccagcagctaaccatctcctggtttgccatcgttttgctggtgtctccactcatggctagcatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagcagtacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 24 - DNA, 1189 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 25 - protein, 10 aa, Artificial Sequence
  linker peptide
GSGSGSGSGS

SEQ ID NO: 26 - DNA, 30 bp, Artificial Sequence
  polynucleotide encoding linker peptide
ggatcaggcagtggttcaggatcaggtagt

(19) United States
(12) Patent Application Publication
(10) Pub. No.: US 2019/0231862 A1
(43) Pub. Date: Aug. 1, 2019

(54) Gene vaccine for preventing and treating severe fever with thrombocytopenia syndrome

(71) Applicant: SLBiGen, Inc., Gyeonggi-do (KR)

(72) Inventors: Hyun-Tak Jin, Gyeonggi-do (KR); Yong Bok Seo, Seoul (KR); Ju-A Shin, Seongnam-si (KR)

(21) Appl. No.: 16/161,189

(22) Filed: Oct. 16, 2018

Related U.S. Application Data
(60) Provisional application No. 62/572,832, filed on Oct. 16, 2017.

(51) Int. Cl. A61K 39/12; C12N 7/00; A61K 38/20; A61K 38/48; A61K 38/27; A61K 39/39; A61P 31/14; C07K 14/005

(52) U.S. Cl. CPC A61K 39/12; C12N 7/00; A61K 38/208; A61K 38/482; A61K 2039/53; A61K 38/27; A61K 39/39; A61P 31/14; C07K 14/005; C12Y 304/21068

(57) ABSTRACT

Provided is a gene vaccine composition for preventing and treating severe fever with thrombocytopenia syndrome (SFTS), including, as an active ingredient, at least one expression vector including a first polynucleotide encoding a glycoprotein

N (Gn) derived from SFTS virus, a second polynucleotide encoding a glycoprotein C (Gc), a third polynucleotide encoding a nucleocapsid protein (NP) derived from the SFTS virus, and a fourth polynucleotide encoding a nonstructural protein (NS) derived from the SFTS virus.

### CROSS REFERENCE TO RELATED APPLICATION

This application claims priority benefit under 35 U.S.C. § 119(e) of U.S. Provisional Application No. 62/572,832 filed on Oct. 16, 2017 and the entire contents of which are incorporated herein by reference.

### BACKGROUND

The present disclosure relates to a gene vaccine, and more particularly, to a gene vaccine for preventing and treating severe fever with thrombocytopenia syndrome virus infection.

Severe fever with thrombocytopenia syndrome (hereinafter, abbreviated as ‘SFTS’) is an acute febrile disease caused by SFTS virus infection, primarily caused by a tick bite during outdoor activities between April and November. The SFTS virus consists of three RNA genome segments: L segment of 6,368 bp, M segment of 3,378 bp, and S segment of 1,744 bp, and a nucleoprotein (NP), a nonstructural protein (NS), an RNA dependent RNA polymerase (RdRp), and glycoproteins C and N (GcGn) corresponding to capsid proteins, the latter part being bound to the RNA genome (see FIG. 1). Ticks that transmit the SFTS virus are presumed to be hard ticks such as Haemaphysalis longicornis and the like. Main initial symptoms are fever and symptoms of the digestive system such as inappetence, diarrhea, nausea, and the like, and patients with dyspnea, mental deterioration, and gastrointestinal tract hemorrhage are at increased risk of serious illness. In South Korea, since the first patient was reported in 2012, by 2015, there were 172 confirmed cases, of which 55 deaths occurred, and thus, SFTS is a very dangerous infectious disease with a mortality rate of 32%. However, diagnostic kits of some viruses, effective specific therapeutic agents, and preventive vaccines have not been reported to date. WO2015053455A1 discloses an isolated SFTS virus and an immunogenic composition including all or part of the SFTS virus, that is, a vaccine, however, it does not present a specific vaccine composition which can prevent actual SFTS infection or treat SFTS.

### SUMMARY

As described above, no effective vaccine composition for preventing SFTS virus infection and treating SFTS has been reported so far. In order to solve various issues including the above one, the present disclosure provides preparation of a gene vaccine composition for preventing SFTS virus infection and treating SFTS.

However, the present disclosure is not limited thereto.

In accordance with an exemplary embodiment of the present disclosure, a gene vaccine composition for preventing and treating severe fever with thrombocytopenia syndrome (SFTS) may include as an active ingredient at least one expression vector including a first polynucleotide encoding glycoproteins N and C (GnGc) derived from SFTS virus, a second polynucleotide encoding a nucleocapsid protein (NP) derived from the SFTS virus, and a third polynucleotide encoding a nonstructural protein (NS) derived from the SFTS virus.

### BRIEF DESCRIPTION OF THE DRAWINGS

FIG. 1 is a schematic diagram showing the structures of SFTS virus and the SFTS virus genome.

FIG. 2A is a schematic diagram showing the structures of three SFTS plasmid DNAs in accordance with an embodiment of the present disclosure; FIG. 2B is a schematic diagram showing the structures of gene constructs, contained in a single SFTS plasmid DNA in accordance with an embodiment of the disclosure, in which polynucleotides encoding Gn-IRES-Gc, NS-NP, and IL-12M, are operably linked to CMV promoter, RSV promoter and EF1α promoter, respectively; FIG. 2C is a schematic diagram showing the structures of the gene constructs, contained in a single SFTS plasmid DNA of the present disclosure, in which polynucleotides encoding GnGc, NP, and NS proteins, are operably linked to CMV promoter, RSV promoter and EF1a promoter, respectively; and FIG. 2D is a schematic diagram showing the structure of the gene construct, contained in a SFTS plasmid DNA expressing a fusion protein in accordance with one embodiment of the present disclosure, in which a polynucleotide encoding GnGc protein is operably linked to CMV promoter, and a polynucleotide prepared in order that NP and NS proteins are expressed in a form of a fusion protein is operably linked to RSV promoter.

FIG. 3A is a graph illustrating the expression level of NP, which is an antigen of the SFTS virus, when the DNA vaccine in accordance with an embodiment of the present disclosure is transfected into COS-7 cells; FIG. 3B is a graph illustrating the expression level of NS, which is an antigen of the SFTS virus, when the DNA vaccine in accordance with an embodiment of the present disclosure is transfected into COS-7 cells; FIG. 3C is a graph illustrating the expression level of GnGc, which is an antigen of the SFTS virus, when the DNA vaccine in accordance with an embodiment of the present disclosure is transfected into COS-7 cells; FIG. 3D is a graph illustrating the results of measuring the expression levels of Flt3L (left) and mIL-12 in experimental animals inoculated with the SFTS single gene vaccine composition in accordance with an embodiment of the present disclosure; and FIG. 3E is a series of photographs illustrating Western blot analysis results for examining the expression levels of Flt3L and IL12 in the experimental animals.

FIG. 4 is a schematic diagram illustrating an administration schedule of the gene vaccine composition in accordance with an embodiment of the present disclosure.

FIG. 5 is a series of graphs illustrating antigen-specific T cell responses in experimental animals administered with the gene vaccine composition in accordance with an embodiment of the present disclosure.

FIG. 6A is a schematic diagram illustrating the administration schedule of the gene vaccine composition for verifying the antibody-inducing effect of the SFTS single gene vaccine composition in accordance with an embodiment of the present disclosure; FIG. 6B is a graph illustrating the result of examining the antibody response according to the dilution ratio to measure the titer of anti-NP antibody response in animals inoculated with the vaccine composition according to the above administration schedule; and FIG. 6C is a graph illustrating the result of calculating the titer of anti-NP IgG from the result of FIG. 6B.

FIG. 7A is a schematic diagram illustrating the administration schedule of the SFTS single gene vaccine composition in accordance with an embodiment of the present disclosure for analyzing the infection-preventing effect of the SFTS single gene vaccine composition in accordance with an embodiment of the present disclosure; FIG. 7B is a graph illustrating changes in weight over time of experimental animals inoculated with the vaccine according to the schedule of FIG. 7A and infected with the SFTS virus; FIG. 7C is a graph illustrating the survival rate of the experimental animals in FIG. 7B over time.

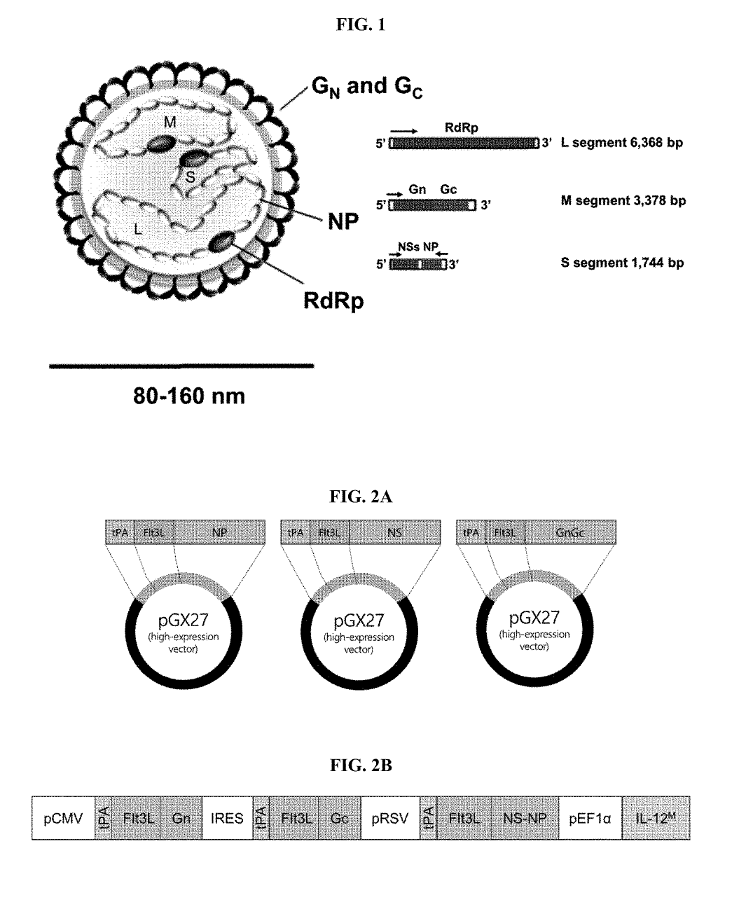

### DETAILED DESCRIPTION OF EMBODIMENTS

In accordance with one aspect of the present disclosure, the provided is a gene vaccine composition for preventing and treating severe fever with thrombocytopenia syndrome (SFTS), including, as an active ingredient, at least one expression vector including a first polynucleotide encoding a glycoprotein N (Gn) derived from SFTS virus, a second polynucleotide encoding a glycoprotein C (Gc) derived from SFTS virus, a third polynucleotide encoding a nucleocapsid protein (NP) derived from the SFTS virus, and a fourth polynucleotide encoding a nonstructural protein (NS) derived from the SFTS virus.

The gene vaccine composition may include any one or more selected from the group consisting of the followings: i) a first expression vector including the first polynucleotide, a second expression vector including the second polynucleotide, a third expression vector including the third polynucleotide, and a fourth expression vector including the fourth polynucleotide; ii) a fifth expression vector including a gene construct in which the first polynucleotide and the second polynucleotide are linked, a sixth expression vector including a gene construct in which the third polynucleotide and the fourth polynucleotide are linked; iii) a seventh expression vector including both a first gene construct in which the first polynucleotide and the second polynucleotide are linked to a first promoter and a second gene construct in which the third polynucleotide and the fourth polynucleotide are sequentially linked to a second promoter; iv) an eighth expression vector including a gene construct in which at least two polynucleotides among the first to the fourth polynucleotides are linked to an internal ribosomal entry site (IRES); and v) a ninth expression vector including a gene construct in which at least two polynucleotides among the first to the fourth polynucleotides are linked to a polynucleotide encoding a linker and which is expressed in a form of a fusion protein.

The expression vectors may include a gene construct in which the polynucleotides are operably linked to a promoter.

The vaccine composition may further include a third gene construct in which a polynucleotide encoding IL-12 is operably linked to a third promoter and the third gene construct may be contained in any one or more of the first to the eighth expression vectors or may be provided by a separate expression vector.

As used herein, the term “operably linked to” means that the nucleic acid sequence of interest (e.g., in an in vitro transcription/translation system or in a host cell) is linked to the regulatory sequence in such a way that the nucleic acid can be expressed.

The term “regulatory sequence” is meant to include promoters, enhancers, and other regulatory elements (e.g., polyadenylation signals). Regulatory sequences include those which direct the constant expression of a nucleic acid of interest in various host cells, those which direct the expression of a nucleic acid of interest only in a specific tissue cell (e.g., tissue-specific regulatory sequences), and those which direct the expression to be induced by a particular signal (e.g., inducible regulatory sequences). Those skilled in the art may appreciate that the design of the expression vector may vary depending on factors such as the selection of a host cell to be transformed and the desired level of protein expression, etc. The expression vector of the present disclosure may be introduced into a host cell to express the fusion protein. Regulatory sequences which enable expressions in eukaryotic and prokaryotic cells are well known to those skilled in the art. As described above, these usually include regulatory sequences responsible for initiating transcription and, optionally, poly-A signals responsible for terminating transcription and stabilizing transcripts. Additional regulatory sequences may include translation-enhancing factors and/or naturally-associated or heterologous promoter regions in addition to transcriptional regulatory elements. For example, possible regulatory sequences enabling expression in mammalian host cells include CMV-HSV thymidine kinase promoter, SV40, RSV-promoter (Rous sarcoma virus), human elongation factor 1α-promoter, the glucocorticoid-inducible MMTV-promoter (Moloney Mouse Tumor Virus), metallothionein-inducible or tetracyclin-inducible promoters, or enhancers, such as CMV enhancer or SV40-enhancer. For expression in neural cells, it is envisaged that neurofilament-, PGDF-, NSE-, PrP-, or thy-1-promoters may be employed. Such promoters are known in the art and described in Charron, J. Biol. Chem. 1995, 270: 25739-25745. For the expression in prokaryotic cells, a number of promoters including the lac-promoter, the tac-promoter, or the trp promoter, are disclosed. In addition to factors capable of initiating transcription, the regulatory sequences may also include transcription termination signals, such as the SV40-poly-A site or the TK-poly-A site, downstream of the polynucleotide in accordance with one embodiment of the present disclosure. In this document, suitable expression vectors are well-known in the art, such as Okayama-Berg cDNA expression vector pcDV1 (Pharmacia), pRc/CMV, pcDNA1, pcDNA3 (Invitrogene), pSPORT1 (GIBCO BRL), pGX27 (Korean Patent No. 1442254), pX (Pagano et al., Science, 255, 1144-1147, 1992), yeast two-hybrid vectors such as pEG202 and dpJG4-5 (Gyuris et al., Cell 75, 791-803, 1995) or prokaryotic expression vectors such as lambda gtl1 or pGEX (Amersham-Pharmacia). In addition to the nucleic acid molecules of the present disclosure, the vector may further include polynucleotide encoding a secretion signal sequence. Such signal sequences are well-known to those skilled in the art. Furthermore, depending on the expression system used, leader sequences capable of directing the fusion protein to a cellular compartment are combined to the coding sequence of the polynucleotide in accordance with an embodiment of the present disclosure, and preferably a leader sequence capable of directly secreting a translated protein or a protein thereof into the periplasmic space or extracellular medium.

In addition, the expression vectors included in the gene vaccine composition of the present disclosure may be prepared by, for example, standard recombinant DNA techniques, and the standard recombinant DNA techniques include, for example, blunt end and adhesive end ligations, restriction enzyme treatment to provide appropriate ends, dephosphorylation by alkaline phosphatase treatment to prevent inadequate binding, and enzymatic linkage by T4 DNA ligase, etc. The vector of the present disclosure may be prepared by recombining the DNA encoding the signal peptide obtained by chemical synthesis or genetic recombinant technology and the DNA encoding the SFTS antigen protein of the present disclosure with a vector including an appropriate regulatory sequence. The vector including the regulatory sequence may be purchased or manufactured commercially, and in an embodiment of the present disclosure, pGX27, which is a vector for producing a DNA vaccine, was prepared and used.

The expression vector may not include a polynucleotide encoding an RNA-dependent RNA polymerase (RdRP).

In the gene vaccine composition, the glycoprotein N (Gn) may have amino acid sequence of SEQ ID NO: 11; a polynucleotide encoding the Gn may have nucleic acid sequence of SEQ ID NO: 12; the glycoprotein C (Gc) may have amino acid sequence of SEQ ID NO: 14; and a polynucleotide encoding the Gc may have nucleic acid sequence of SEQ ID NO: 15. In addition, the Gn and the Gc may be expressed in a form of a fusion protein (GnGc protein) in which two are linked as one and in this case, the GnGc protein may have amino acid sequence of SEQ ID NO: 1 and a polynucleotide encoding the GnGc protein may have nucleic acid sequence of SEQ ID NO: 4.

In the gene vaccine composition, a polynucleotide encoding the NP may have nucleic acid sequence of SEQ ID NO: 5, and a polynucleotide encoding the NS may have nucleic acid sequence of SEQ ID NO: 6. Likewise, the NP and the NS may be expressed in a form of a fusion protein (NP-NS protein) and in this case, the NP-NS protein may be in a form linked by a linker peptide or directly.

In addition, in the gene vaccine composition of the present disclosure, the SFTS virus-derived antigen protein may be known variants, in particular, variants derived from the SFTS virus isolated from South Korea. For example, the GnGc variant protein may be derived from the strains described in Table 1 below, the NP variant proteins may be derived from the strains described in Table 2 below, and the NS variant protein may be derived from the strains described in Table 3 below.

TABLE 1
GnGc protein variants derived from the
SFTS virus isolated from South Korea

| | | Variation | |
|---|---|---|---|
| Strain name | Homology | location | Reference |
| KACNH3 | 100% | N/A | Yun et al., |
| KAGBH6 | 99% | M2I, Y83F, | Genome Announc. |
| | | M499I, V904I, | 3(2): e00181-15, 2015 |
| | | T905S, V1065A | |
| KAGBH5 | 99% | Y83F, I477V, | |
| | | V903I | |
| KAGWH3 | 99% | Y83F, V108I, | |
| | | N524V, V903I, | |
| | | K1060R | |
| KASJH | 99% | T21S, N37S, | |
| | | I457L, L469I, | |
| | | S501T, K577R, | |
| | | V904I, S1192T | |
| Gangwon/Korea/ | 99% | L13F, T21S, | Kim et al., |
| 2012 | | N37D, S218G, | Emerg. Infect. Dis., |
| | | G300E, P341Q, | 19(11): 1892-1894, |
| | | V479I, M491I, | 2013 |
| | | G562S, K577R, | |
| | | S662P, V904I, | |
| | | L1053M, S1192T | |

TABLE 2
NP protein variants derived from the
SFTS virus isolated from South Korea

| | | Variation | |
|---|---|---|---|
| Strain name | Homology | location | Reference |
| JP07-Korea-14 | 100% | N/A | Yun et al., Am. |
| JP07-Korea-13 | 99% | Q14H | J. Trop. Med. Hyg., |
| JP06-Korea-13 | 99% | V230I | 93(3): 468-474, 2015 |
| JP03-Korea-13 | 99% | T19S, R52K, | |
| | | I230V | |
| KASJH | 99% | T19S, R52K | |
| Gangwon/Korea/ | 99% | R52K, A156T | Kim et al., |
| 2012 | | | Emerg. Infect. Dis., |
| | | | 19(11): 1892-1894, 2013 |

TABLE 3
NS protein variants derived from the
SFTS virus isolated from South Korea

| | | Variation | |
|---|---|---|---|
| Strain name | Homology | location | Reference |
| JP07-Korea-14 | 100% | N/A | Yun et al., Am. |
| JP07-Korea-13 | 99% | H72Q, V286I | J. Trop. Med. Hyg., |
| JP06-Korea-13 | 99% | V286I | 93(3): 468-474, 2015 |
| JP06-Korea-14 | 99% | H72Q, V286I | |
| JP05-Korea-14 | 99% | H72Q, V286I | |
| JP05-Korea-13 | 99% | H72Q, V286I | |
| JP04-Korea-14 | 99% | H72Q, V286I | |
| JP04-Korea-13 | 99% | H72Q, V286I | |
| JP03-Korea-14 | 99% | H72Q, V286I | |
| JP03-Korea-13 | 99% | H72Q, V286I | |
| JP02-Korea-14 | 99% | H72Q, V286I | |
| JP02-Korea-13 | 99% | H72Q, V286I | |
| JP01-Korea-14 | 99% | H72Q, V286I | |
| JP01-Korea-13 | 99% | H72Q, V286I | |
| DP01-Korea-13 | 98% | H72Q, M235L, | |
| | | I243V, R281K, | |
| | | V286I | |
| CP01-Korea-13 | 98% | N17S, H72Q, | |
| | | V234I, M235L, | |
| | | I243V, V286I | |
| AP01-Korea-13 | 98% | H72Q, M235L, | |
| | | I243V, V286I | |
| KACNH3 | 98% | H72Q, R210C, | Yun et al., Genome |
| | | M235L, E237D, | Announc. 3(2): |
| | | I243V, V286I | e00181-15, 2015 |
| KAGBH6 | 98% | H72Q, M235L, | |
| | | I243V, R281K, | |
| | | V286I | |
| KAGBH5 | 99% | H72Q, M235L, | |
| | | V286I | |
| KAGWH3 | 99% | H72Q, M235L, | |
| | | I243V, V286I | |
| KASJH | 97% | C6S, H72Q, | |
| | | R130K, V234I, | |
| | | M235L, I243 A, | |
| | | Y249H, V286I | |
| Gangwon/Korea/ | 97% | H72Q, Q144R, | Kim et al., Emerg. |
| 2012 | | E170D, M235L, | Infect. Dis., 19(11): |
| | | I243V, V286I, | 1892-1894, 2013 |
| | | I289P | |

In the gene vaccine composition, at least two among the GnGc, the NP, and the NS may be expressed in a form of a fusion protein or may be individually expressed by an internal ribosome entry site (IRES) or a separate promoter.

As used herein, the term “fusion protein” refers to a recombinant protein in which two or more proteins or domains responsible for a specific function within a protein are linked so that each protein or domain is responsible for its intrinsic function. A linker having a flexible structure may conventionally be inserted between the two or more proteins or domains. Various linkers such as GS₄ are known as such linkers.

The expression vector may further include a polynucleotide encoding one or at least two immunity-enhancing peptides, and the immunity-enhancing peptides may be a cytoplasmic domain of CD28, inducible costimulator (ICOS), cytotoxic T lymphocyte associated protein 4 (CTLA4), programmed cell death protein 1 (PD1), B and T lymphocyte associated protein (BTLA), death receptor 3 (DR3), 4-1BB, CD2, CD40, CD30, CD27, signaling lymphocyte activation molecule (SLAM), 2B4 (CD244), natural-killer group 2, member D (NKG2D)/DNAX-activating protein 12 (DAP12), T-Cell immunoglobulin and mucin domain containing protein 1 (TIM1), TIM2, TIM3, TIGIT, CD226, CD160, lymphocyte activation gene 3 (LAG3), B7-1, B7-H1, glucocorticoid-induced TNFR family related protein (GITR), fms-like tyrosine kinase 3 (Flt3) ligand, flagellin, herpesvirus entry mediator (HVEM), or OX40L [ligand for CD134 (OX40), CD252], or a fusion protein of two or more thereof.

The expression vector may further include a polynucleotide encoding a secretion signal sequence, and the secretion signal sequence induces the extracellular secretion of the SFTS antigen proteins and may be a signal sequence for tissue plasminogen activator (tPA), a signal sequence for herpes simplex virus glycoprotein Ds (HSV gDs), or a signal sequence for growth hormone.

In addition, the polynucleotide included in the gene vaccine composition in accordance with an embodiment of the present disclosure may be substituted with a codon having a high expression frequency in a host cell. As used herein, “one substituted with a codon having a high expression frequency in a host cell” or “optimized codon” mean that, when DNA is transcribed and translated into a protein in a host cell, there are codons having high preference according to the host, among the codons that direct amino acids, and that the expression efficiency of the amino acid or protein encoded by the nucleic acid is increased by substituting with codons having high preference.

The gene vaccine composition in accordance with an embodiment of the present disclosure may include an expression vector which allows the expression of the above three antigen proteins in a host cell. The expression vector may be in any form, including a plasmid vector, a viral vector, a cosmid vector, a phagemid vector, an artificial human chromosome, etc.

The gene vaccine composition may include a pharmaceutically acceptable carrier and/or adjuvant.

In addition to the carrier, the gene vaccine composition may further include a pharmaceutically acceptable adjuvant, excipient, or diluent.

As used herein, the term “pharmaceutically acceptable” refers to a composition that is physiologically acceptable and does not normally cause an allergic reaction, such as a gastrointestinal disorder and dizziness, or similar reactions, when administered to humans. Examples of the carrier, excipient, and diluent may include lactose, dextrose, sucrose, sorbitol, mannitol, xylitol, erythritol, maltitol, starch, acacia gum, alginate, gelatin, calcium phosphate, calcium silicate, cellulose, methylcellulose, polyvinylpyrrolidone, water, methyl hydroxybenzoate, propyl hydroxybenzoate, talc, magnesium stearate, and mineral oil. In addition, fillers, anti-coagulants, lubricants, humectants, fragrances, emulsifiers, preservatives, etc., may be additionally included.

In the gene vaccine composition, the adjuvant may be aluminum hydroxide, aluminum phosphate, alum (potassium aluminum sulfate), MF59, virosome, AS04 [a mixture of aluminum hydroxide and monophosphoryl lipid A (MPL)], AS03 (a mixture of DL-α-tocopherol, squalene, and polysorbate 80 which is an emulsifier), CpG, Flagellin, Poly I:C, AS01, AS02, ISCOMs, or ISCOMMATRIX.

As used herein, the term “adjuvant” refers to a pharmaceutical or immunological agent that is administered for the purpose of enhancing the immune response of a vaccine. The adjuvant may be used such as aluminum hydroxide, aluminum phosphate, alum (potassium aluminum sulfate), MF59, virosome, AS04 [a mixture of aluminum hydroxide and monophosphoryl lipid A (MPL)], AS03 (a mixture of DL-α-tocopherol, squalene, and polysorbate 80 which is an emulsifier), CpG, Flagellin, Poly I:C, AS01, AS02, ISCOMs, or ISCOMMATRIX.

In addition, the gene vaccine composition in accordance with an embodiment of the present disclosure may be formulated using a method known in the art to allow rapid release, or sustained or delayed release of an active ingredient upon its administration to a mammal. Formulations include powders, granules, tablets, emulsions, syrups, aerosols, soft or hard gelatin capsules, sterile injectable solutions, and sterile powders.

The gene vaccine composition in accordance with an embodiment of the present disclosure may be administered by various routes including, for example, oral, parenteral, e.g., suppository, transdermal, intravenous, intraperitoneal, intramuscular, intralesional, intranasal, and intraspinal routes, and may be administered using an implantable device for sustained or continuous or repeated release. Administrations may be carried out once or several times a day within a desired range, and the administration period is not particularly limited.

The gene vaccine composition in accordance with one embodiment of the present disclosure may be administered by a conventional systemic or local administration method, e.g., intramuscular injection or intravenous injection, and most preferably it may be injected by means of an electroporator. The electroporator to be used may include an electric perforator for injecting commercially-available DNA drugs into the body, e.g., Glinporator™ (IGEA, Italy), CUY21EDIT (JCBIO, South Korea), SP-4a (Supertech, Switzerland), etc.

With regard to the administration route, the gene vaccine composition in accordance with an embodiment of the present disclosure may be administered via any conventional route as long as it can reach a tissue of interest. Such administration route may be, but not limited to, parenteral routes, for example, intraperitoneal, intravenous, intramuscular, subcutaneous, and intrasynovial routes.

The gene vaccine composition in accordance with an embodiment of the present disclosure may be formulated in a suitable form together with a commonly used pharmaceutically acceptable carriers. Pharmaceutically acceptable carriers may include, for example, water, suitable oils, saline solution, carriers for parenteral administration such as aqueous glucose and glycols, and the like, and the gene vaccine composition may further include a stabilizer and a preservative. Examples of suitable stabilizers are antioxidants such as sodium hydrogen sulfite, sodium sulfite, and ascorbic acid. Examples of suitable preservatives are benzalkonium chloride, methyl- or propyl-paraben, and chlorobutanol. Furthermore, when necessary according to the administration method or formulations, the composition in accordance with the present disclosure may appropriately include a suspending agent, a solubilizer, a stabilizer, an isotonic agent, a preservative, an adsorption inhibitor, a surfactant, a diluent, an excipient, a pH adjuster, an analgesic agent, a buffering agent, an antioxidant, etc. Pharmaceutically acceptable carriers and preparations suitable for the present disclosure, including those exemplified above, are described in detail in Remington's Pharmaceutical Sciences, recent edition.

The dosage for a patient of the gene vaccine composition varies depending on many factors, including the patient's height, body surface area, age, a particular compound to be administered, gender, time and route of administration, general health conditions, and other drugs to be administered simultaneously. Pharmaceutically active DNA may be administered in an amount of 100 ng/body weight (kg) to 10 mg/body weight (kg), more preferably 1 μg/kg to 500 μg/kg (body weight), and most preferably 5 μg/kg to 50 μg/kg (body weight), and may be administered in a unit dose of 10 μg, 100 μg, 1 mg, 2 mg, 3 mg, 4 mg, 5 mg, 10 mg, 20 mg, 30 mg, 40 mg, 50 mg, 60 mg, 70 mg, 80 mg, 90 mg, or 100 mg, and dosages may be adjusted considering the above factors.

Discovery of antigen proteins based on immunoproteomics is to search antigen proteins that cause immune responses actually in virus-infected patients and thus it is possible to prepare highly feasible vaccines based on the searched proteins. Thus, the present inventors conducted an analysis based on immunoproteomics through prior studies in order to find an immunogenic antigen which plays an important role in the prevention and treatment of various antigens of the SFTS virus in blood of patients with SFTS virus infection. However, the results showed that immunogenicity in patients with SFTS virus infection was not concentrated on a specific antigen protein or a specific domain of an antigen, but, on the contrary, it was found that there were immune responses against various domains of various antigens such as GnGc, NP, NS, or the like. Based on these results, it was found that it is more appropriate to develop a vaccine in a form of a plasmid DNA which can induce the overall immune responses against various antigens and various domains of the antigens rather than to prepare a vaccine against a specific antigen or a specific domain of the concerned antigen.

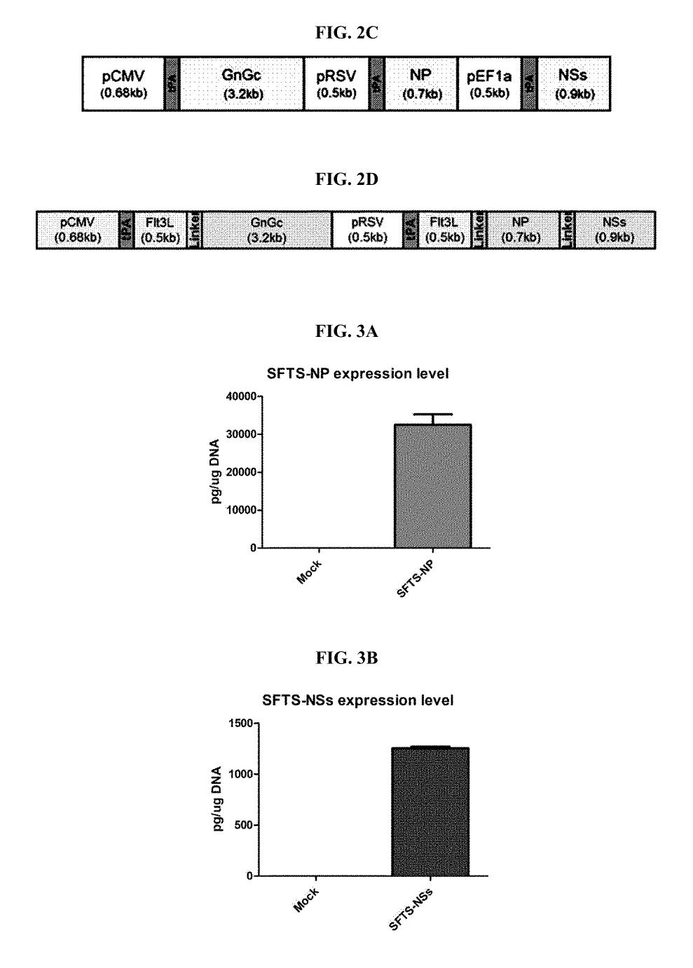

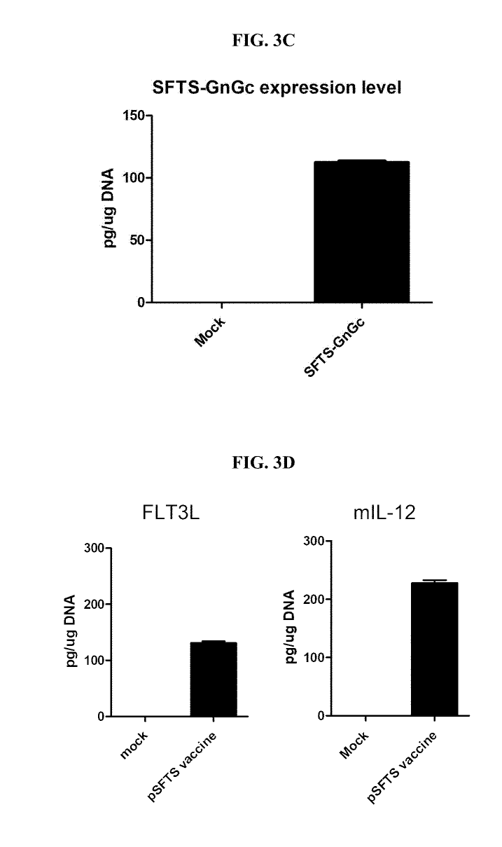

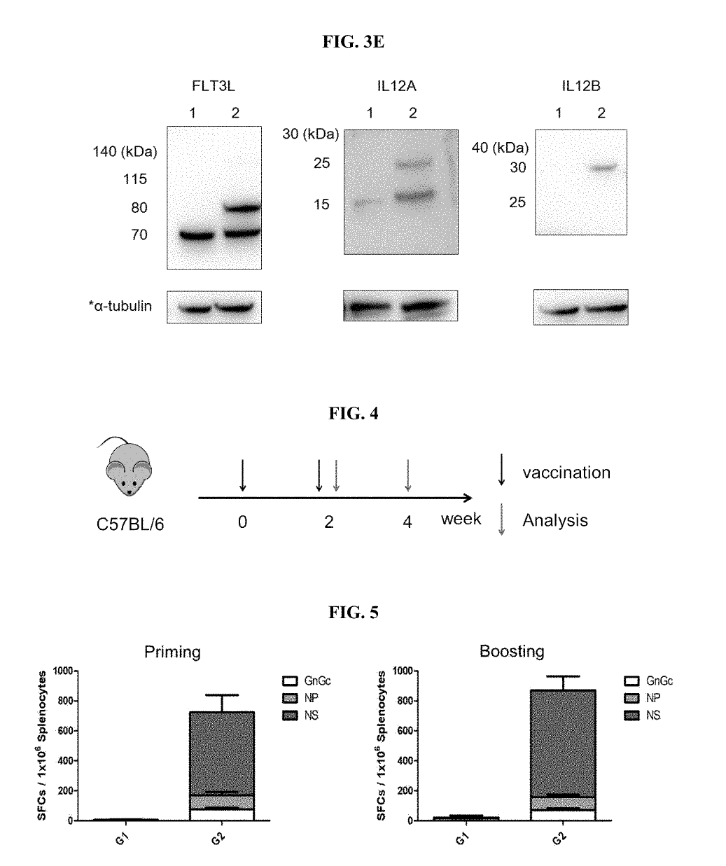

Thus, the present inventors found that when preparing three plasmid DNAs for expressing SFTS antigens, which were constructed so as to express the GnGc, the NP, and the NS, respectively, except the RdRp, among antigen proteins of the SFTS virus (see FIG. 2A), and transfecting them into mammalian cells, the three plasmid DNAs could express effectively the antigen proteins (see FIGS. 3A to 3E) and when combining the three SFTS plasmid DNAs and inoculating experimental animals, the combined three SFTS plasmid DNAs could induce antigen-specific immune responses (see FIG. 5), thus completing the present disclosure. In an embodiment of the present disclosure, all of the three antigen proteins were cloned into individual expression vectors and expressed. However, the three antigen proteins may be contained in one expression vector and expressed, or may be divided into two expression vectors and expressed. When two or more antigen proteins are cloned into one expression vector, the antigen proteins may be expressed in a polycistronic manner, by operably linking each of the antigen proteins to a separate promoter to insert them, or by linking the antigen proteins to a polynucleotide encoding a linker peptide so as to express the antigen proteins in a form of a fusion protein, or by linking polynucleotides encoding each of the antigen proteins to an internal ribosome entry site (IRES).

### MODES FOR CARRYING OUT THE INVENTION

Hereinafter, the present disclosure is explained in more detail through Examples and Experimental examples. However, the present disclosure is not limited to these Examples and Experimental examples described below, but may be implemented in various other forms, and the following Examples and Experimental examples are provided to fully illustrate the scope of the present disclosure to those of ordinary skill in the art to which the present disclosure pertains.

### EXAMPLE 1

### Preparation of SFTS Plasmid DNAs

In order to express the SFTS virus antigens, the GnGc (glycoproteins N and C, SEQ ID NO: 1), the NP (nucleocapsid, SEQ ID NO: 2), the NS (nonstructural protein, SEQ ID NO: 3) antigens, except the RdRP (RNA dependent RNA polymerase) antigen, in a vector, the present inventors constructed three recombinant plasmid DNAs (SFTS plasmid DNAs), by synthesizing polynucleotides encoding each of the proteins (SEQ ID NOs: 4 to 6) (Cosmogenetech, Genscript), and then linking them to polynucleotide (SEQ ID NO: 8) encoding tissue plasminogen activator (tPA) secretion signal sequence (SEQ ID NO: 7) as a secretion signal and polynucleotide (SEQ ID NO: 10) encoding Flt3L(SEQ ID NO: 9), and then inserting them into the polycloning site of pGX27 plasmid DNA (WO2012020871A1) (FIG. 2A).

### EXAMPLE 2

### Preparation of Single SFTS Plasmid DNA in which Gn and Gc are Separated

The present inventors slightly modified the DNA construct of Example 1 and prepared a single SFTS plasmid DNA which enables Gn and Gc to express in a separated form by IRES.

Specifically, a Gn gene construct was prepared by linking polynucleotide (SEQ ID NO: 12) encoding the Gn polypeptide (SEQ ID NO: 11) following the polynucleotide (SEQ ID NO: 8) encoding tPA secretion signal sequence (SEQ ID NO: 7) as a secretion signal and the polynucleotide (SEQ ID NO: 10) encoding Flt3L (SEQ ID NO: 9), and then operably linking it to CMV promoter (SEQ ID NO: 13). Then, a Gc gene construct in which, similarly to the Gn gene construct, polynucleotide (SEQ ID NO: 15) encoding the Gc polypeptide (SEQ ID NO: 14) was linked instead of the polynucleotide encoding the Gn polypeptide, was linked to the internal ribosome entry site (IRES) having nucleic acid sequence of SEQ ID NO: 16. In addition, a NS-NP gene construct was prepared by linking polynucleotide (SEQ ID NO: 18) encoding the NS-NP polypeptide (SEQ ID NO: 17), which enabled the NS polypeptide and the NP polypeptide to express in a form of a fusion protein, following polynucleotide encoding tPA-Flt3L, and then operably linking it to RSV promoter (SEQ ID NO: 19) and the NS-NP gene construct was linked following the Gc gene construct. Lastly, a gene construct, in which polynucleotides (SEQ ID NOs: 22 and 23) encoding the respective mIL-12A (SEQ ID NO: 20) and mIL-12B (SEQ ID NO: 21) were linked by the IRES (SEQ ID NO: 16), was operably linked to EF1α promoter (SEQ ID NO: 24) and then, the gene construct was linked to the NS-NP gene construct (see FIG. 2B). The multiple gene construct in which four gene constructs were linked into one as described above was inserted into the pGX27 vector.

### EXAMPLE 3

### Preparation of Single SFTS Plasmid DNA

A single SFTS plasmid DNA is prepared by preparing a gene construct, in which the polynucleotide (SEQ ID NO: 8) encoding tPA secretion signal sequence (SEQ ID NO: 7), the polynucleotide (SEQ ID NO: 4) encoding the GnGc (SEQ ID NO: 1), the RSV promoter (SEQ ID NO: 19), the polynucleotide (SEQ ID NO: 8) encoding tPA secretion signal sequence, the polynucleotide (SEQ ID NO: 5) encoding the NP (SEQ ID NO: 2), the EF1α promoter (SEQ ID NO: 24), the polynucleotide (SEQ ID NO: 8) encoding tPA secretion signal sequence, and the polynucleotide (SEQ ID NO: 6) encoding the NS (SEQ ID NO: 3) are sequentially linked, and then inserting it to the polycloning site of pGX27 vector (FIG. 2C).

### EXAMPLE 4

### Preparation of SFTS Plasmid DNA Expressing a Fusion Protein

A SFTS plasmid DNA designed to express the NP and the NS in a form of a fusion protein with the GnGc protein is prepared by preparing a gene construct in which the polynucleotide (SEQ ID NO: 8) encoding tPA secretion signal sequence (SEQ ID NO: 7), the polynucleotide (SEQ ID NO: 10) encoding Flt3L (SEQ ID NO: 9), the polynucleotide (SEQ ID NO: 26) encoding the linker peptide (SEQ ID NO: 25), the polynucleotide (SEQ ID NO: 4) encoding the GnGc (SEQ ID NO: 1), the RSV promoter (SEQ ID NO: 19), the polynucleotide (SEQ ID NO: 8) encoding tPA secretion signal sequence, the polynucleotide (SEQ ID NO: 10) encoding Flt3L (SEQ ID NO: 9), the polynucleotide (SEQ ID NO: 26) encoding the linker peptide (SEQ ID NO: 25), the polynucleotide (SEQ ID NO: 5) encoding the NP (SEQ ID NO: 2), the polynucleotide (SEQ ID NO: 26) encoding the linker peptide (SEQ ID NO: 25), and the polynucleotide (SEQ ID NO: 6) encoding the NS (SEQ ID NO: 3) are sequentially linked, and then inserting it to the polycloning site of pGX27 vector (FIG. 2D).

### EXPERIMENTAL EXAMPLE 1

### Examination of SFTS Plasmid DNA Products

The present inventors examined whether the inserted gene products were expressed in the SFTS plasmid DNAs in accordance with an embodiment of the present disclosure prepared in Example 1 above. Specifically, the COS-7 cell line was inoculated onto a 100 mm culture dish and cultured for 16 hours. Then, the COS-7 cells were transfected with an empty vector (mock plasmid DNA) and three SFTS plasmid DNAs, respectively, using Lipofectamine 2000, and cultured in a 37° C. CO₂ incubator for 3 days. Culture supernatants of COS-7 cells under each condition were then collected and used as samples. Since proteins present in the sample were in the form in which Flt3L was bound, quantification was carried out using an Flt3L ELISA kit (FIGS. 3A to 3C).

As a result, as shown in FIGS. 3A to 3C, the expression amounts of proteins in samples into which the SFTS plasmid DNA was introduced were 32,481 pg/μg DNA for the NP protein, 1,254 pg/μg DNA for the NS protein, and 112 pg/μg DNA for the GnGc protein, and the expression amount of protein for the mock plasmid DNA was below the quantitative limit.

In addition, the present inventors examined whether the inserted gene products were expressed rightly in the single SFTS plasmid DNA in accordance with an embodiment of the present disclosure prepared in Example 2 above. Specifically, the COS-7 cell line was inoculated onto a 100 mm culture dish and cultured for 16 hours. Then, the COS-7 cells were transfected with an empty vector (mock plasmid DNA) and the SFTS plasmid DNA prepared in Example 2 using Lipofectamine 2000, and cultured in a CO2 cell incubator under a temperature condition of 37° C. for 3 days. Culture supernatants of COS-7 cells were then collected and used as samples. Since proteins present in the sample were Flt3L-bound proteins or mIL-12, quantification was carried out using an Flt3L ELISA kit or a mIL-12 ELISA kit. As a result, as shown in FIG. 3D, the expression amount of antigen proteins in the sample into which the single SFTS plasmid DNA was introduced was 131.1 pg/μg DNA, and the expression amount of mIL-12 was 227.8 pg/μg DNA.

In addition, 2×106 cells of COS-7 cells were transfected with the SFTS plasmid in a 100 mm culture dish using Lipofectamine 2000, and cultured in a 37° C. incubator for 2 days. Then, COS-7 cells were collected, and placed on ice to undergo lysis. The supernatant obtained by centrifugation was then measured for protein concentration using a BCA assay kit and transferred to a 1.5 mL tube so that the total protein amount was 70 μg. After addition of a SDS sample buffer and a reducing buffer, proteins in the sample were denatured at 72° C. for 10 minutes and subjected to SDS gel electrophoresis. By using PVDF, proteins were transferred to a membrane. Then, a first antibody (anti-FLT3L, anti-mIL-12 antibody) which is specific for a gene product was treated and then, a HRP-conjugated secondary antibody was treated. Color was developed by ECL solution and the inserted gene product was detected. As a result, as shown in FIG. 3E, a specific band was observed at the predicted molecular weight of 70-80 kDa of the SFTS antigen fusion proteins (FLT3L-Gn, FLT3L-Gc, and FLT3L-NP-NS), and we examined proteins corresponding to predicted molecular weights of 28 kDa and 37 kDa by using the IL-12p35- and IL-p40-specific antibody, but no specific protein was detected in the negative control.

### EXPERIMENTAL EXAMPLE 2

### Examination of In Vivo Immunogenicity by Administration of the SFTS Plasmid DNA

In order to examine whether the SFTS plasmid DNA in accordance with an embodiment of the present disclosure exhibits significant in vivo immunogenicity, the present inventors evaluated the immune responses to the corresponding antigens after co-administration of the above three plasmid DNAs.

Specifically, C57/BL6 mice were divided into a control group that is administered with an empty vector (mock plasmid DNA) and an experimental group that is administered with three SFTS plasmid DNAs, respectively. 12 μg of the corresponding plasmid DNA was administered intramuscularly by in vivo electroporation delivery method two times at 2-week intervals into each administration group (FIG. 4). Two weeks after the final administration, the immune responses were evaluated using ex vivo IFN-γ ELISPOT assays (FIG. 5).

TABLE 4
Administration dose and administration route of
the plasmid DNA vaccine of the present disclosure

| | | The number of | | Route |
|---|---|---|---|---|
| | | experimental | Dose | (administration |
| Group | Vaccine | animals | (μg) | method) |
| Control | mock plasmid | 3 | 12 | intramuscular |
| group | | | | (electroporation) |
| Experimental | SFTS plasmid | 5 | 12 | intramuscular |
| group | DNA | | | (electroporation) |

As a result, as shown in FIG. 5, it was found that significant antigen-specific T cell responses were induced in the group administered with all of the three SFTS plasmid DNAs compared to the control group after priming and boosting.

### EXPERIMENTAL EXAMPLE 3

### In Vivo Antibody Response by Administration of the SFTS Plasmid DNA

In addition, the present inventors investigated whether the single SFTS plasmid DNA vaccine prepared in Example 2 above caused immune responses against SFTS through animal experiments.

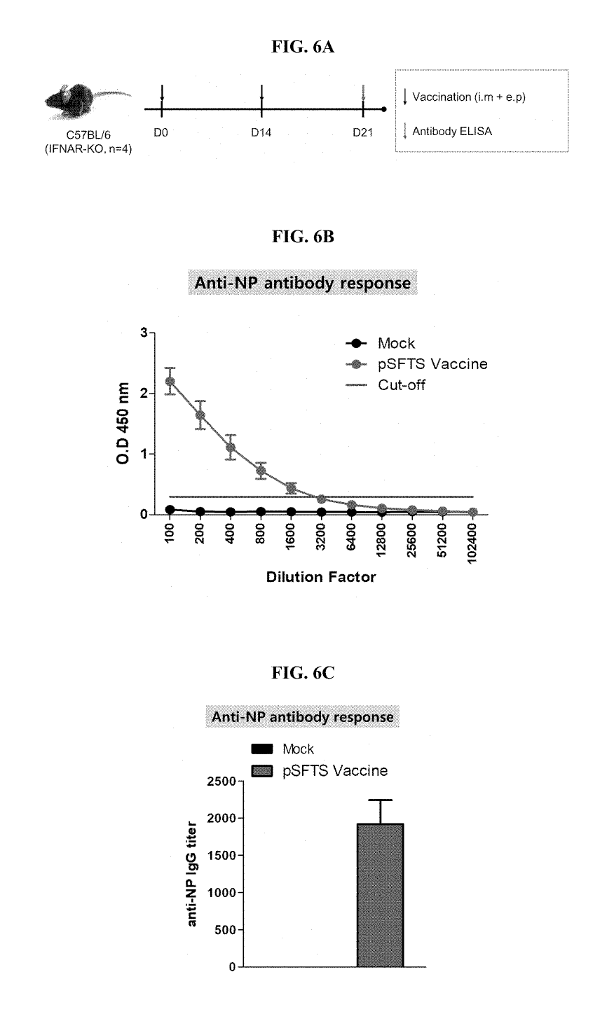

To this end, specifically, C57/BL6 mice were divided into a control group that is administered with an empty vector (mock plasmid DNA) and an experimental group that is administered with the single SFTS plasmid DNA prepared in Example 2, respectively (4 animals per group). 12 μg of the corresponding plasmid DNA was administered intramuscularly by in vivo electroporation delivery method two times at 2-week intervals into each administration group (FIG. 6A). One week after the final administration, anti-NP antibody was quantified via sandwich ELISA assay (FIGS. 6B and 6C). As a result, as shown in FIGS. 6B and 6C, it could be found that the single SFTS plasmid DNA in accordance with an embodiment of the present disclosure could effectively induce the production of the antibody specific for the NP polypeptide, which is the main antigen protein of SFTS.

### EXPERIMENTAL EXAMPLE 4

### Analysis of the Preventing Effect of the SFTS DNA Vaccine

The present inventors investigated whether the single SFTS plasmid DNA vaccine in accordance with an embodiment of the present disclosure had the preventing effect on SFTS infection.

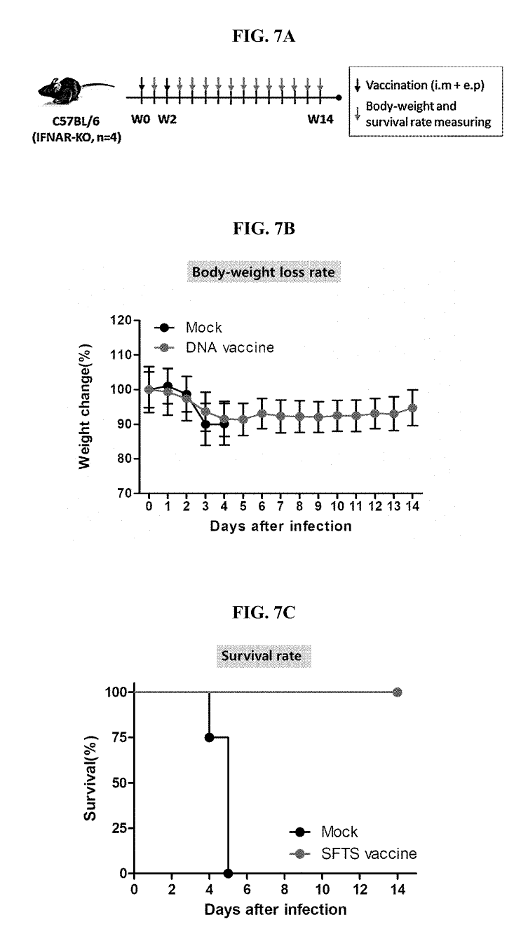

To this end, specifically, interferon-alpha (IFNAR) knockout C57/BL6 mice were divided into two groups and a control group (n=4) was administered with a mock plasmid DNA and an experimental group (n=4) was administered with 12 μg of the single SFTS plasmid DNA prepared in Example 2, intramuscularly by in vivo electroporation delivery method two times (FIG. 7A). After mice were infected with SFTS virus, the weights (FIG. 7B) and the survival rate (FIG. 7C) of the experimental animals were measured until 14 weeks after the first vaccination. SFTS virus infection was performed four weeks after the day of the first vaccination (two weeks after the day of the second vaccination). As a result, as shown in FIGS. 7B and 7C, when the SFTS gene vaccine composition in accordance with an embodiment of the present disclosure was administered to interferon-alpha knockout mice, the body weights were somewhat reduced, but all the experimental animals survived until the end of the experiment. Meanwhile the experimental animals not administered with the SFTS gene vaccine composition in accordance with an embodiment of the present disclosure died within 4 to 5 weeks of infection. This is the result showing that the SFTS DNA vaccine in accordance with an embodiment of the present disclosure can effectively prevent the SFTS infection.

As described above, when administered in vivo, the SFTS plasmid DNA in accordance with an embodiment of the present disclosure could efficiently induce the T-cell-specific immune response to the SFTS antigen. In addition, on vaccination in advance, the SFTS plasmid DNA in accordance with an embodiment of the present disclosure could mitigate symptoms of SFTS infection, thereby exhibiting the excellent preventing effect on SFTS. Therefore, the SFTS plasmid DNA in accordance with an embodiment of the present disclosure can be used very efficiently for the preparation of DNA vaccines for preventing SFTS virus infection and treating SFTS.

While the present disclosure has been described with reference to previously described Examples and Experimental examples, it is only for illustrative purposes. It will be understood by those skilled in the art that various modifications and equivalent other embodiments may be made thereto. Therefore, the scope of the true technical protection of the present disclosure should be determined by the technical idea of the appended claims.

1. A gene vaccine composition for preventing and treating severe fever with thrombocytopenia syndrome (SFTS), comprising, as an active ingredient, at least one expression vector including a first polynucleotide encoding a glycoprotein N (Gn) derived from SFTS virus, a second polynucleotide encoding a glycoprotein C (Gc) derived from SFTS virus, a third polynucleotide encoding a nucleocapsid protein (NP) derived from the SFTS virus, and a fourth polynucleotide encoding a nonstructural protein (NS) derived from the SFTS virus.

2. The gene vaccine composition of claim 1, comprising any one or more selected from the group consisting of the followings:
i) a first expression vector including the first polynucleotide, a second expression vector including the second polynucleotide, a third expression vector including the third polynucleotide, and a fourth expression vector including the fourth polynucleotide;
ii) a fifth expression vector including a gene construct in which the first polynucleotide and the second polynucleotide are linked, a sixth expression vector including a gene construct in which the third polynucleotide and the fourth polynucleotide are linked;
iii) a seventh expression vector including both a first gene construct in which the first polynucleotide and the second polynucleotide are linked to a first promoter and a second gene construct in which the third polynucleotide and the fourth polynucleotide are sequentially linked to a second promoter;
iv) an eighth expression vector including a gene construct in which at least two polynucleotides among the first to the fourth polynucleotides are linked to an internal ribosomal entry site (IRES); and
v) a ninth expression vector including a gene construct in which at least two polynucleotides among the first to the fourth polynucleotides are linked to a polynucleotide encoding a linker and which is expressed in a form of a fusion protein.

3. The gene vaccine composition of claim 1, wherein the expression vector does not include a polynucleotide encoding an RNA-dependent RNA polymerase (RdRP).

4. The gene vaccine composition of claim 1, further comprising a third gene construct in which a polynucleotide encoding IL-12 is operably linked to a third promoter.

5. The gene vaccine composition of claim 4, wherein the third gene construct is contained in any one or more of the first to the eighth expression vectors or is provided by a separate expression vector.

6. The gene vaccine composition of claim 1, wherein the glycoprotein Gn has amino acid sequence of SEQ ID NO: 11.

7. The gene vaccine composition of claim 1, wherein the glycoprotein Gc has amino acid sequence of SEQ ID NO: 14.

8. The gene vaccine composition of claim 1, wherein the NP has amino acid sequence of SEQ ID NO: 2.

9. The gene vaccine composition of claim 1, wherein the NS has amino acid sequence of SEQ ID NO: 3.

10. The gene vaccine composition of claim 1, wherein the polynucleotide encoding the glycoprotein N has nucleic acid sequence of SEQ ID NO: 12.

11. The gene vaccine composition of claim 1, wherein the polynucleotide encoding the glycoprotein C has nucleic acid sequence of SEQ ID NO: 15.

12. The gene vaccine composition of claim 1, wherein the polynucleotide encoding the NP has nucleic acid sequence of SEQ ID NO: 5.

13. The gene vaccine composition of claim 1, wherein the polynucleotide encoding the NS has nucleic acid sequence of SEQ ID NO: 6.

14. The gene vaccine composition of claim 1, wherein the expression vector further comprises a polynucleotide encoding one or at least two immunity-enhancing peptide.

15. The gene vaccine composition of claim 14, wherein the immunity-enhancing peptide is a cytoplasmic domain of CD28, inducible costimulator (ICOS), cytotoxic T lymphocyte associated protein 4 (CTLA4), programmed cell death protein 1 (PD1), B and T lymphocyte associated protein (BTLA), death receptor 3 (DR3), 4-1BB, CD2, CD40, CD30, CD27, signaling lymphocyte activation molecule (SLAM), 2B4 (CD244), natural-killer group 2, member D (NKG2D)/DNAX-activating protein 12 (DAP12), T-Cell immunoglobulin and mucin domain containing protein 1 (TIM1), TIM2, TIM3, TIGIT, CD226, CD160, lymphocyte activation gene 3 (LAG3), B7-1, B7-H1, glucocorticoid-induced TNFR family related protein (GITR), fms-like tyrosine kinase 3 (Flt3) ligand, flagellin, herpesvirus entry mediator (HVEM), or OX40L [ligand for CD134(OX40), CD252], or a connection of two or more thereof.

16. The gene vaccine composition of claim 1, wherein the expression vector further comprises a polynucleotide encoding a secretion signal sequence.

17. The gene vaccine composition of claim 16, wherein the secretion signal sequence is a signal sequence for tissue plasminogen activator (tPA), a signal sequence for herpes simplex virus glycoprotein Ds (HSV gDs), or a signal sequence for growth hormone.

18. The gene vaccine composition of claim 1, further comprising a pharmaceutically acceptable carrier and/or adjuvant.

19. The gene vaccine composition of claim 18, further comprising a pharmaceutically acceptable excipient and/or diluent.

20. The gene vaccine composition of claim 18, wherein the adjuvant is aluminum hydroxide, aluminum phosphate, alum (potassium aluminum sulfate), MF59, virosome, AS04 [a mixture of aluminum hydroxide and monophosphoryl lipid A (MPL)], AS03 (a mixture of DL-a-tocopherol, squalene, and polysorbate 80 which is an emulsifier), CpG, Flagellin, Poly I: C, AS01, AS02, ISCOMs, or ISCOMMATRIX.
